(19) United States
(12) Patent Application Publication
(10) Pub. No.: US 2006/0224115 A1
(43) Pub. Date: Oct. 5, 2006

(54) Balloon catheter with expandable wire lumen

(75) Inventor: Martin R. Willard, Burnsville, MN (US)

Correspondence Address: VIDAS, ARRETT & STEINKRAUS, P.A., 6109 BLUE CIRCLE DRIVE, SUITE 2000, MINNETONKA, MN, 55343-9185, US

(73) Assignee: Boston Scientific Scimed, Inc., Maple Grove, MN (US)

(21) Appl. No.: 11/093,556

(22) Filed: Mar. 30, 2005

(51) Int. Cl. A61M 29/00

(52) U.S. Cl. 604/103.04

(57) ABSTRACT

A catheter comprises an outer shaft and an inner shaft defining a balloon inflation lumen for transport of an inflation fluid therethrough. The expandable balloon has a proximal waist portion and a distal waist portion with a body portion there between. The balloon is engaged to a distal region of the outer shaft and defines an exterior and an interior where the interior is in fluid communication with the balloon inflation lumen. The inner shaft has an inner shaft wall defining a guidewire lumen for passage of a guidewire. A distal portion of the inner shaft is expandable from an unexpanded state to an expanded state such that in the expanded state the guidewire lumen has a greater cross-sectional area than in the unexpanded state.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

This invention embodies such areas as those related to catheters and catheter assemblies for use in medical procedures. In more specific embodiments, this invention relates to catheter systems, including those used in percutaneous transluminal angioplasty (PTA), cryoplasty and/or cooling procedures, etc.

2. Description of the Related Art

Percutaneous transluminal angioplasty (PTA) is a procedure which is well established for the treatment of blockages, lesions, stenosis, thrombus, etc. present in body lumens such as the peripheral or coronary arteries and/or other vessels.

A widely used form of percutaneous transluminal angioplasty makes use of a dilatation balloon catheter which is introduced into and advanced through a lumen or body vessel until the distal end thereof is at a desired location in the vasculature. Once in position across an afflicted site, the expandable portion of the catheter, or balloon, is inflated to a predetermined size with a fluid at relatively high pressures. By doing so the vessel is dilated, thereby radially compressing the atherosclerotic plaque of any lesion present against the inside of the artery wall, and/or otherwise treating the afflicted area of the vessel. The balloon is then deflated to a small profile so that the dilatation catheter may be withdrawn from the patient's vasculature and blood flow resumed through the dilated artery.

It is known that in some angioplasty procedures, the reopening of a vessel is in whole or in-part frustrated by complete or partial reclosure of the artery or vessel. Often the mechanism responsible for the closure of the vessel is vessel recoil and/or more commonly restenosis of the lesion resulting from continued growth of the lesion back into the vessel.

In angioplasty procedures of the kind described above, there may be restenosis of the artery, which either necessitates another angioplasty procedure, a surgical by-pass operation, or some method of repairing or strengthening the area. To reduce restenosis and strengthen the area, a physician can implant an intravascular prosthesis for maintaining vascular patency, such as a stent, inside the artery at the lesion.

In some cases, where the vessel and/or surrounding tissue has had its blood flow blocked or reduced, it has been shown that by cooling the tissue the amount of necrosis is reduced if re-profusion is established within a given treatment window. However current catheter systems do not adequately provide both a mechanism for establishing re-profusion and providing a cooling effect within the desired window.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### SUMMARY OF THE INVENTION

In at least one embodiment, the invention is directed to a catheter comprising an outer shaft and an inner shaft. The inner shaft may have an inner shaft wall defining a guidewire lumen for passage of a guidewire. In at least one embodiment, the catheter may further comprise an expandable balloon. In at least one embodiment, the inner and outer shafts may define a balloon inflation lumen for transport of an inflation fluid there through. In at least one embodiment, the expandable balloon may have a proximal waist portion and a distal waist portion with a body portion there between. In at least one embodiment, the balloon may be engaged to a distal region of the at least one outer shaft and may define an exterior and an interior. In at least one embodiment, the interior may be in fluid communication with the balloon inflation lumen. In at least one embodiment, a distal portion of the inner shaft may be expandable. In at least one embodiment, the inner shaft may be constructed and arranged to release infusate onto the surrounding tissue without removal of an extant guidewire. In at least one embodiment the guidewire may help diffuse the infusate as it passes through the guidewire lumen.

In at least one embodiment, a distal portion of the inner shaft may be expandable from an unexpanded state to an expanded state. In at least one embodiment, the inner shaft is constructed of deformable material. In at least one embodiment, in the expanded state the guidewire lumen may have a greater cross-sectional area than in the unexpanded state. In at least one embodiment, a guidewire may be disposed within the guidewire lumen. In at least one embodiment, the guidewire is partially retracted such that the distal most end of the guidewire is proximal to the distal waist portion of the balloon.

In at least one embodiment, the guidewire lumen when in the expanded state is constructed and arranged to permit passage of an infusate through the inner shaft. The infusate can be passed out around the distal end of the catheter.

In at least one embodiment, the distal portion of the guidewire lumen may have an expanded position and an unexpanded position such that when in the expanded position infusate can more easily attain a sufficient infusate flow rate through the guidewire lumen than when in the unexpanded position.

In at least one embodiment, the distal portion of the guidewire lumen may have a diameter of between 0.010 and 0.017 inches when in the unexpanded position.

In at least one embodiment, the distal portion of the guidewire lumen may have a diameter of between 0.018 and 0.025 inches when in the expanded position.

In at least one embodiment, a distal portion of the inner shaft may be constructed of a foldable material that will unfold with infusate pressure. In at least one embodiment, the foldable material is ePTFE.

In at least one embodiment, a distal portion of the inner shaft is constructed of an elastic or flexible material that radially stretches with infusate pressure. In at least one embodiment, the elastic material may be an elastomeric material. In at least one embodiment, the elastomeric material may include, though not be limited to, elastomeric polyurethane, elastomeric block copolymers including the styrenic block copolymers such as styrene-ethylene/butylene-styrene (SEBS) block copolymers disclosed in U.S. Pat. No. 5,112,900 which is incorporated by reference herein in its entirety. Other suitable block copolymer elastomers include, but are not limited to, styrene-isoprene-styrene (SIS), styrene-butadiene-styrene (SBS), styrene-isobutylene-styrene (SIBS), styrene-ethylene/propylene-styrene (SEPS) and so forth. Block copolymer elastomers are also described in commonly assigned U.S. Pat. Nos. 6,406,457, 6,171,278, 6,146,356, 5,951,941, 5,830,182, 5,556,383, each of which is incorporated by reference herein in its entirety.

Elastomeric polyesters and copolyesters may also be employed herein. Examples of elastomeric copolyesters include, but are not limited to, poly(ester-block ether) elastomers, poly(ester-block-ester) elastomers and so forth. Poly(ester-block-ether) elastomers are available under the tradename of HYTREL® from DuPont de Nemours & Co. and consist of hard segments of polybutylene terephthalate and soft segments based on long chain polyether glycols. Such polymers are also available from DSM Engineering Plastics under the tradename of ARNITEL®.

Non-elastomeric materials may also be employed herein. Examples of non-elastomeric materials include, but are not limited to, polyolefins, polyesters, polyethers, polyamides, polyurethanes, polyimides, copolymers and terpolymers thereof, and so forth. As used herein, the term “copolymer” shall be hereinafter be used to refer to any polymer formed from two or more monomers. Non-elastomeric polyesters and copolymers thereof such as polyalkylene naphthalates including polyethylene terephthalate and polybutylene terephthalate may also be used.

Polyamides including nylon, and copolymers thereof may be employed herein. Block copolymer elastomers such as poly(ether-block-amides) may be employed herein and are available from Atofina Chemicals in Philadelphia, Pa., under the tradename of PEBAX®.

In other embodiments, any combination of the above elastic or elastomeric materials may be used.

In at least one embodiment, the distal portion of the inner shaft which is expandable may be constructed of a material that shortens as it expands radially. In at least one embodiment, the distal portion of the inner shaft which is expandable may be constructed of a mesh having an expanding cover which separates the mesh from the balloon inflation lumen. In at least one embodiment, longitudinal pressure applied distally to a proximal portion of the inner shaft may radially expand the distal portion of the inner shaft which is expandable.

In at least one embodiment, the inner shaft may be affixed to the outer shaft at a point distal to the body portion of the balloon such that beyond this point the inner shaft does not substantially move in relation to the outer shaft.

In at least one embodiment, the expandable distal portion of the inner shaft may be constructed and arranged to expand proximal to the proximal waist portion of the balloon and within the outer shaft.

In at least one embodiment, the expandable distal portion of the inner shaft may be constructed and arranged to expand distal to the proximal waist portion of the balloon.

In at least one embodiment, the inner shaft may be attached to the distal waist portion of the balloon. In at least one embodiment, the distal waist portion may have coolant ports constructed and arranged to allow release of an infusate.

In at least one embodiment, the infusate may be a fluid having a temperature below about 37 Celsius.

In at least one embodiment, at least a portion of the distal waist portion may be an extension of the inner shaft. In at least one embodiment, at least a portion of the distal waist portion may have coolant ports which allow release of an infusate.

In at least one embodiment, the coolant ports may be constructed and arranged such that infusate will not substantially pass through the coolant ports before the expandable portion of the inner shaft is expanded.

In at least one embodiment, the infusate may be a fluid having a temperature of about 33 degrees Celsius to about 37 degrees Celsius.

In at least one embodiment, at least a portion of the distal waist portion may comprise a plurality of coolant ports or openings. In at least one embodiment, the ports may be constructed and arranged to allow the guidewire lumen during expansion to build pressure while allowing a sufficient outflow of infusate to adequately cool the surrounding tissue.

In at least one embodiment, the infusate has a viscosity less than that of blood.

In some embodiments the infusate comprises a solution of one or more fluids such as saline, Ringer Lactate solution etc.

In at least one embodiment, coolant ports are configured to allow the infusate to exit the guidewire lumen under pressure but prevent or restrict the flow of bodily fluids into the guidewire lumen during deflation.

In some embodiments the catheter may be configured for the delivery of one or more therapeutic agents. In at least one embodiments a therapeutic agent is included with the infusate.

These and other embodiments which characterize the invention are pointed out with particularity in the claims annexed hereto and forming a part hereof. However, for a better understanding of the invention, its advantages and objectives obtained by its use, reference should be made to the drawings which form a further part hereof and the accompanying descriptive matter, in which there is illustrated and described a embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a partial cross-sectional side view of an embodiment of the invention.

FIG. 2 is a cross-sectional view along view A-A of the embodiment shown in FIG. 1 showing a foldable guidewire lumen.

FIG. 3 is a partial cross-sectional side view of the catheter having a substantially inflated balloon and a substantially uninflated guidewire lumen.

FIG. 4 is a partial cross-sectional side view of the catheter having a substantially uninflated balloon and a substantially uninflated guidewire lumen.

FIG. 5 is a partial cross-sectional side view of the catheter having a partially inflated balloon and a substantially inflated guidewire lumen.

FIG. 5a is a partial cross-sectional side view of the catheter having a partially inflated balloon and a substantially uninflated guidewire lumen.

FIG. 5b is a partial cross-sectional side view of the catheter having a partially inflated balloon and a substantially inflated guidewire lumen delivering infusate.

FIG. 5c is a partial cross-sectional side view of the catheter having a fully inflated balloon and a substantially inflated guidewire lumen.

FIG. 6 is a partial cross-sectional side view of the catheter having a partially inflated balloon and a substantially inflated guidewire lumen.

FIG. 7 is a partial cross-sectional side view of the catheter showing a more proximal manifold.

FIG. 8 is a partial cross-sectional side view of an embodiment of the invention wherein the guidewire lumen beneath the balloon is relatively unexpanded.

FIG. 9 is a partial cross-sectional side view of an embodiment of the invention wherein the guidewire lumen beneath the balloon is relatively expanded.

FIG. 10 is a partial cross-sectional side view of an embodiment of the invention wherein the inner shaft is attached to the balloon cone.

FIG. 11 is a partial cross-sectional side view of an embodiment of the invention wherein the inner shaft is attached to the balloon cone and unexpanded.

FIG. 11a is a partial cross-sectional side view of an embodiment of the invention wherein the inner shaft is attached to the balloon cone and expanded.

FIG. 12 is a partial cross-sectional side view of an embodiment of the invention wherein the inner shaft is attached to the balloon cone and unexpanded.

FIG. 12a is a partial cross-sectional side view of an embodiment of the invention wherein the inner shaft is attached to the balloon cone and expanded.

FIG. 13 is a partial cross-sectional side view of an embodiment of the invention wherein the inner shaft is attached to the balloon cone and unexpanded.

FIG. 13a is a partial cross-sectional side view of an embodiment of the invention wherein the inner shaft is attached to the balloon cone and expanded.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

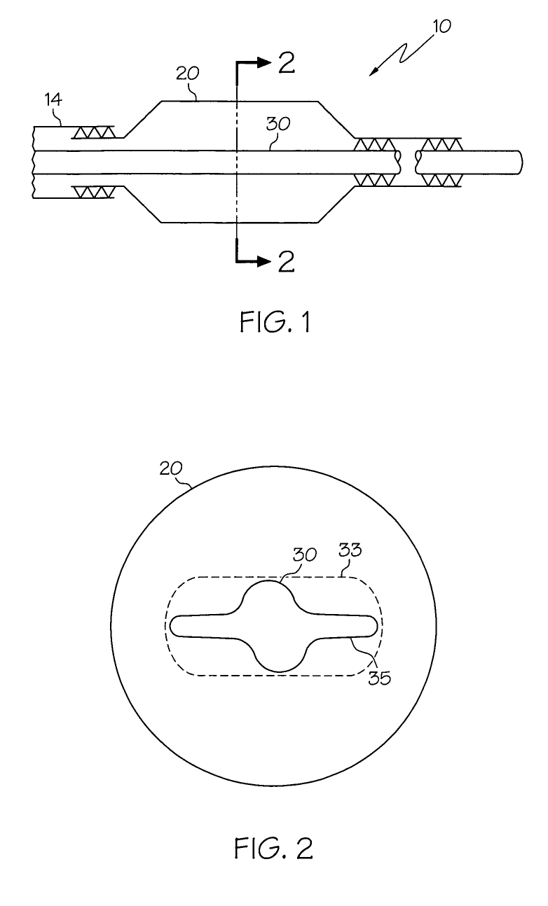

In at least one embodiment, an example of which is shown in FIG. 1, the catheter 10 may have an outer shaft 14, a balloon 20, and an inner shaft 30. In at least one embodiment, the inner shaft may be expandable as illustrated in FIG. 2. FIG. 2 illustrates the cross-section of FIG. 1 along lines A-A. The inner shaft may be in a relatively expanded state 33 or a relatively unexpanded state 35. In the unexpanded state portions of the shaft 30 may fold over on other portions of the shaft. The inner shaft may be self expanding or expandable by interior force. In some embodiments the inner shaft may expand due to structural characteristics; longitudinal distal pressure may shorten the inner shaft while at the same time expanding it.

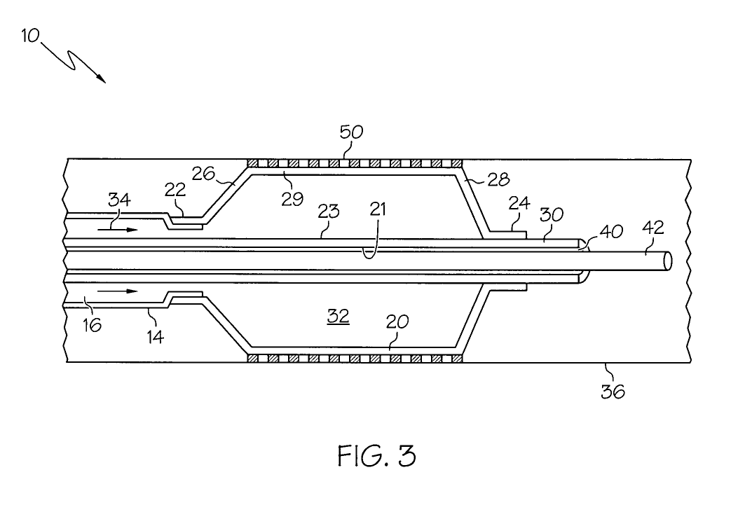

In at least one embodiment as illustrated in FIG. 3, the balloon 20 may be in an inflated state while the inner shaft 30 may be in a relatively unexpanded state. The inner shaft 30 has a shaft inner wall 21 and a shaft outer wall 23. The balloon 20 may include a proximal waist 22, a distal waist 24, a proximal cone 26, a distal cone 28 and a working or body portion 29 therebetween. When mounted on the catheter 10 the proximal waist 22 of the balloon 20 is engaged to a portion of the outer shaft 14 and the distal waist 24 is engaged to a portion of the inner shaft 30. As a result of this configuration the interior 32 of the balloon 20 is in fluid communication with the balloon inflation lumen 16. By transmitting an inflation fluid, indicated by arrow 34, under pressure through the lumen 16, the balloon 20 may be expanded from a collapsed and/or folded reduced diameter configuration to an expanded greater diameter configuration within a body lumen or vessel 36, such as is shown.

The catheter 10, may be a push catheter, over-the-wire catheter, a single operator exchange catheter (e.g. the MONORAIL® catheter available from Boston Scientific Scimed, Inc.), rapid exchange catheter or other type of catheter desired. The inner shaft 30 defines a second lumen or guidewire lumen 40, through which a guidewire 42 may be passed. The catheter 10 may then be advanced along the guidewire 42 to a predetermined location in the vessel 36.

In some embodiments an expandable endoprosthesis such as a stent 50 may be disposed about the balloon, such that when the balloon 20 is expanded the stent is also expanded for delivery into the vessel 36.

As used herein the term ‘stent’ refers to an expandable prosthesis for implantation into a body lumen or vessel and includes devices such as stents, grafts, stent-grafts, vena cava filters, etc. In some embodiments a stent may be at least partially constructed of any of a variety of materials such as stainless steel, nickel, titanium, nitinol, platinum, gold, chrome, cobalt, as well as any other metals and their combinations or alloys. In some embodiments a stent may be at least partially constructed of a polymer material. In some embodiments a stent may be at least partially constructed of a shape-memory polymer or material. In some embodiments a stent may be balloon expandable, self-expandable, hybrid expandable or a combination thereof. In some embodiments a stent or other portions of the catheter may include one or more radiopaque members. In some embodiments a stent may include one or more therapeutic and/or lubricious coatings applied thereto.

In at least one embodiment a removable guidewire 42 may reside within the inner shaft 30. In at least some embodiments the pressure within the balloon interior 32 may inhibit or may prevent the expansion of the inner shaft 30. In at least one embodiment a medical device (e.g. stent) 50 may be disposed about the balloon 20.

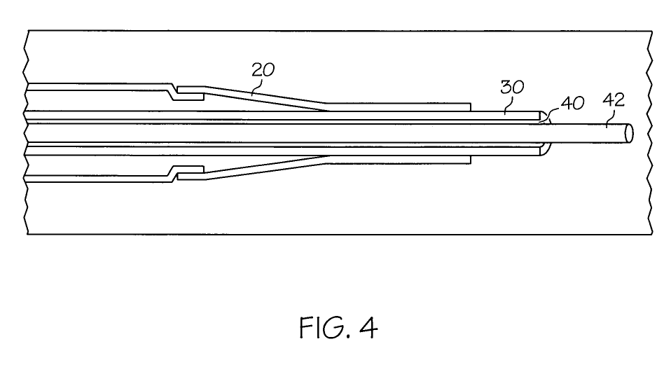

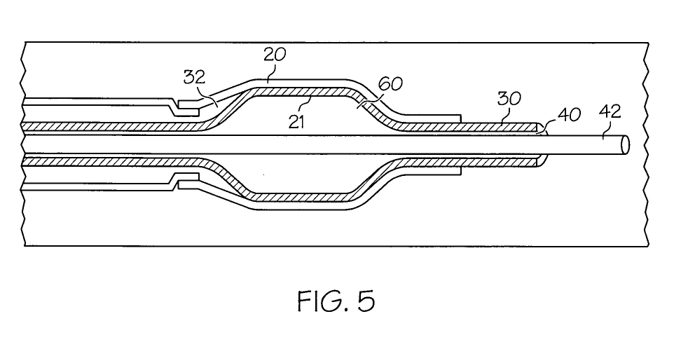

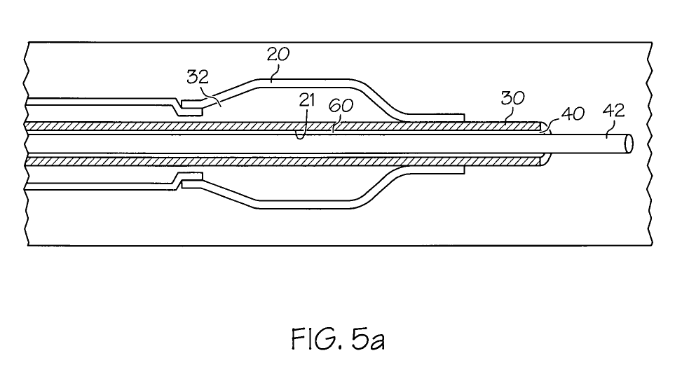

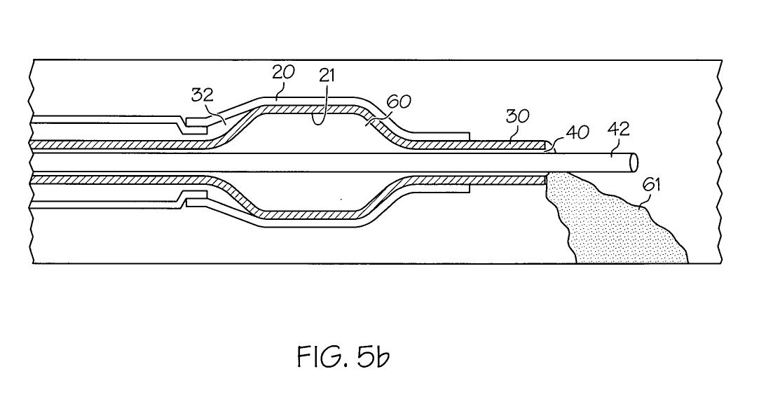

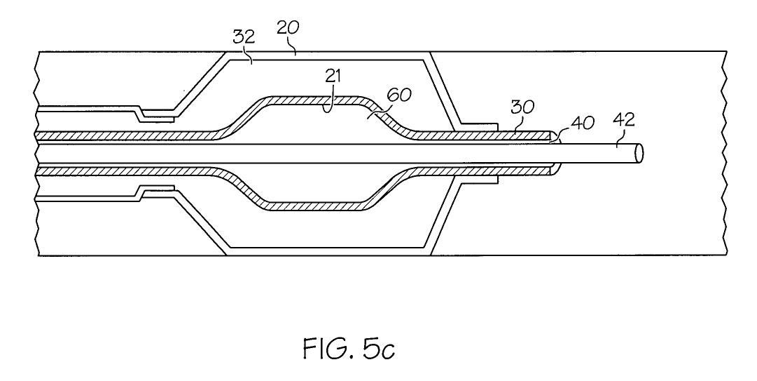

The balloon 20 may be designed such that when the balloon is in an uninflated state the balloon may have a smaller profile than the rest of the catheter as illustrated in FIG. 4. The balloon 20 may be in a collapsed or folded position and the pressure within the balloon interior 32 may be ambient pressure. The balloon may be expanded by transmission of an inflation fluid into the balloon 20 or by force of expansion of the guidewire lumen as shown in FIG. 5. Here, the inner shaft 30 is in an expanded state. A low pressure within the balloon interior 32, such as ambient pressure, allows for easier expansion of the guidewire lumen 60. In at least one embodiment, the balloon 20 is at least partially expanded when the pressure within the balloon interior 32 is above one atmosphere. In at least one embodiment, the balloon may be partially expanded to a diameter of about 2.5 mm which in some embodiments is attained by about ambient pressure within the balloon interior 32. In at least one embodiment, also shown in FIG. 5, the guidewire lumen 60 expands under pressure greater than the elevated pressure or ambient pressure within the balloon interior.

In at least one embodiment, the balloon 20 is partially expanded before the infusate expands the guidewire lumen 60 as shown in FIG. 5a. In at least one embodiment the balloon 20 is partially expanded to 2.5 mm. In at least one embodiment, as shown in FIG. 5b, the infusate/coolant 61 then enters and/or expands the lumen 60 of the inner shaft 30 and can then be released into the vessel lumen 36 through the distal end of the guidewire lumen 60. In at least one embodiment, the release of infusate 61 occurs without the removal of the guidewire 42. In at least one embodiment, the flow into the vessel lumen 36 lasts for about 4 minutes. In some embodiments, the balloon is then fully expanded as shown in FIG. 5c. It should be noted that in some embodiments full expansion of the balloon 20 hinders or prevents inflation of the inner shaft 30 as the pressure within the balloon 20 works against expansion of the inner shaft 30 (alternatively shown in FIG. 3).

The infusate may be a fluid. In at least one embodiment, the fluid is any of a variety of liquid mediums such as saline (with or without additional therapeutic agents), ringers, lactated ringers, etc. In at least one embodiment the fluid is a liquid. In at least one embodiment the infusate is also characterized as a coolant, having been cooled to, or having an inherent temperature of about 37 degrees Celsius or less. In at least one embodiment, the infusate 61 has a temperature of about 33 degrees Celsius to about 36 degrees Celsius.

When the infusate 61 is passed into the lumen 60 and more significantly, as shown in FIG. 5b, is outflowing into the vessel 36 via the distal end 40 of inner shaft 30 or via the coolant ports 90 (illustrated in FIG. 10), the fluid may provide a cooling effect to the surrounding tissues of the vessel 36. This cooling effect may help to reduce additional necrosis of the vessel tissue while blood flow is reduced or stopped during a treatment procedure.

In some embodiments the infusate comprises a therapeutic agent which may be passed into the vessel 36 to treat the surrounding tissues as well as provide the cooling effect previously mentioned.

In some cases a therapeutic agent may be placed in the interior wall of inner shaft 30 or guidewire lumen 60 in the form of a coating that reacts with or is picked up by the infusate as it flows therethrough. The coating/therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic includes cellular material, the cellular material may include but is not limited to: cells of human origin and/or non-human origin as well as their respective components and/or derivatives thereof. Where the therapeutic agent includes a polymer agent, the agent may be a polystyrene-polyisobutylene-polystyrene triblock copolymer (SIBS), polyethylene oxide, silicone rubber and/or any other suitable substrate.

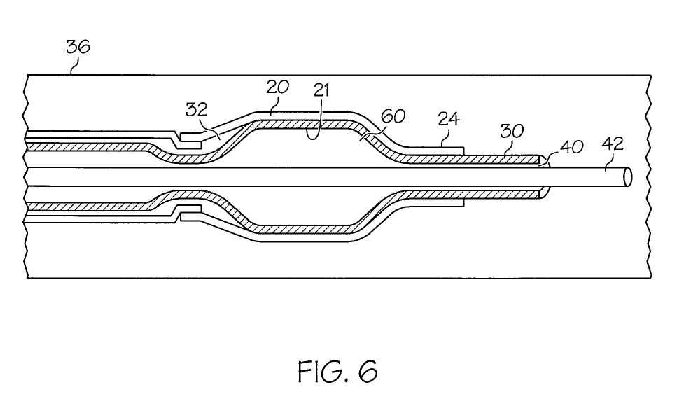

As shown in FIG. 5, the guidewire lumen 60 may expand solely in the area under the balloon. In contrast and as shown in FIG. 6, in at least one embodiment the distal portion of the guidewire lumen 60 which expands includes portions of the guidewire lumen 60 proximal to the balloon. In at least one embodiment, the expanding distal portion of the guidewire lumen 60 includes about one third of the length of guidewire lumen. It should be noted that in some embodiments the guidewire lumen 60 expands after the balloon 20 has partially expanded (as shown in the transition from FIG. 5a to FIG. 5b). Additionally, in some embodiments, the distal end of the inner shaft may be constructed such that infusate passing through the distal end is eliminated or greatly reduced and more infusate passes through holes in the distal waist portion 24 of the balloon 20 and/or in the portion of the inner shaft 30 under and/or distal to the distal waist portion 24 such that infusate may pass into the vessel 36 without passing through the distal most end 40 of inner lumen 30.

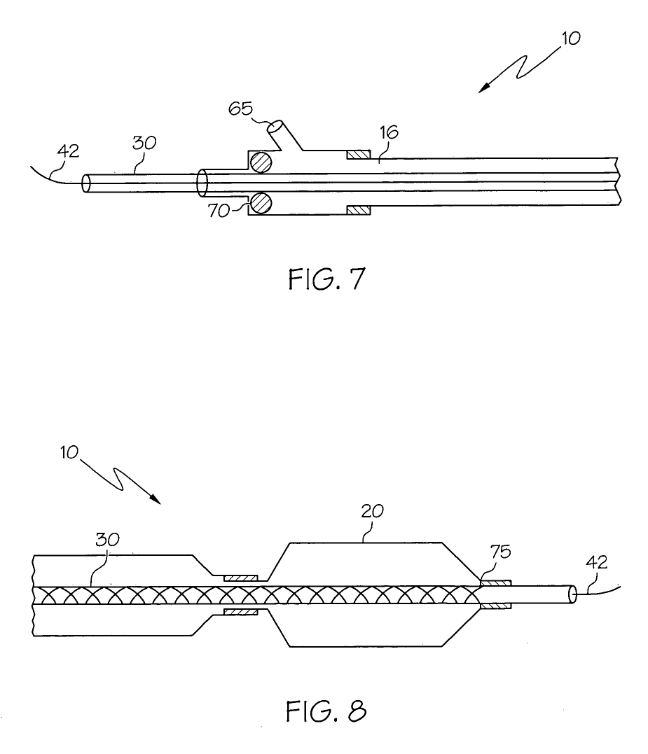

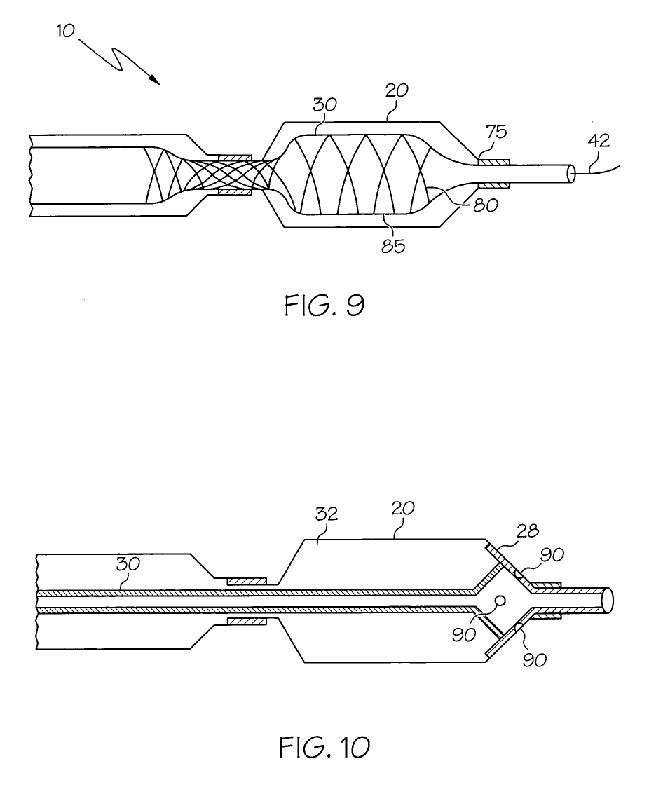

A proximal portion of catheter 10 is shown in FIG. 7. The inner shaft 30 may be slidable within portions of the catheter 10 when axial force is applied to the inner shaft 30 in a distal or proximal direction. In at least one embodiment, the inner shaft 30 may extend proximally in relation to the balloon inflation port 65. Axial force can be applied to the portion of the inner shaft 30 extending proximally at the proximal end of catheter 10. The catheter 10 may be constructed and arranged with seals 70 which seal the fluid introduced through the balloon inflation port 65 from escaping outside the balloon inflation lumen 16. The seals 70 may also be constructed and arranged to allow the inner shaft 30 to slidably move within the catheter while maintaining the seal between the balloon inflation lumen and the portion of the catheter 10 proximal of the seals 70.

As shown in FIG. 8, in at least one embodiment, the slidable inner shaft 30 may be affixed to the balloon 20 at a distal connection 75 such that portions of the inner shaft 30 distal to the connection point 75 may not slide axially in relation to the catheter 10. In at least one embodiment, the inner shaft may be constructed and arranged such that when axial pressure is applied to the inner shaft 30 in a distal direction, portions of the inner shaft proximal of the connection point 75 slide within the catheter 10. In at least one embodiment, the inner shaft 30 is constructed and arranged to shorten and expand when axial pressure is applied to the inner shaft 30 in a distal direction. An unexpanded inner shaft 30 is shown in FIG. 8 in which axial force is not being applied to the inner shaft 30 in a distal direction.

As shown in FIG. 9, when axial pressure is applied to the inner shaft 30 in a distal direction the shaft 30 may expand. This axial pressure is preferably applied at a proximal portion of the inner shaft 30. Because the inner shaft is connected to the balloon at connection point 75, axial pressure applied to the inner shaft 30 in a distal direction compresses the inner shaft 30 proximal to the connection point 75 such that the inner shaft 30 shortens and expands. In at least one embodiment of the invention about a third of the inner shaft 30 shortens and/or expands with axial pressure. In at least one embodiment, a portion of the inner shaft 30 disposed beneath the balloon 20 may be constructed and arranged to substantially expand before other portions of the inner shaft 30. The expansion of the inner shaft 30 may reduce the pressure necessary for maintaining a sufficient flow rate of the infusate into the body lumen 36. When the inner lumen is unexpanded more pressure may be necessary to maintain a sufficient flow of infusate and may result in the undesirable effect of infusate jetting from the catheter 10. It should be noted, that the inner shaft 30 is constructed such that the inner shaft does not significantly shorten or expand while being advanced along the guidewire 42.

In at least one embodiment, the inner shaft 30 is constructed of a braid/mesh 80 which is capable of expanding and shortening with distal longitudinal pressure. The braid/mesh 80 may be fluidly isolated from the balloon inflation media by an expanding cover 85. In at least one embodiment the expanding cover is polyurethane. In at least one embodiment the expanding cover is constructed of an elastomeric material such as those listed above.

In at least one embodiment, the inner shaft 30 is self expanding. In at least one embodiment, the inner shaft 30 is constructed of a braid/mesh 80 which is capable of self-expansion. The braid/mesh 80 may be fluidly isolated from the balloon inflation media by an expanding cover 85 such as polyurethane. In at least one embodiment the expanding cover is an constructed of an elastomeric material such as those listed above.

In at least one embodiment, as shown in FIG. 10, the expandable inner shaft 30 may be attached to the distal cone 28 such that it is in fluid communication with the distal cone 28 and separated from the balloon interior 32. In at least one embodiment the inner shaft 30 and the distal cone 28 are constructed of the same piece of material. In at least one embodiment, the distal cone 28 may have coolant ports 90 through which infusate may be released. The coolant ports may be constructed and arranged to prohibit flow through the ports 90 when the infusate is under a pressure below a predetermined pressure.

In at least one embodiment, the coolant ports 90 are configured to allow the infusate to pass out of the inner lumen 30 when pressurized, but may also be configured to minimize or prevent back flow of fluids, such as blood, from entering the guidewire lumen 60 from the vessel 36 during the application of negative pressure during collapse/refold of the balloon prior to withdrawal of the catheter 10 from the vessel 36.

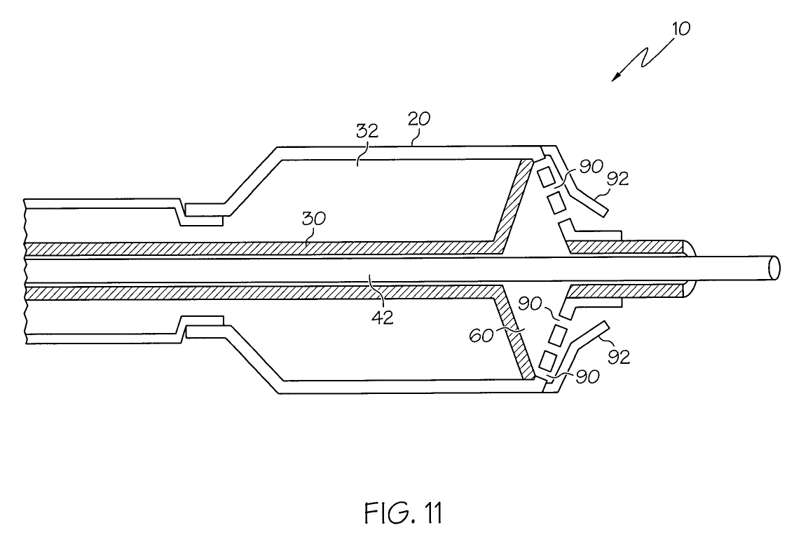

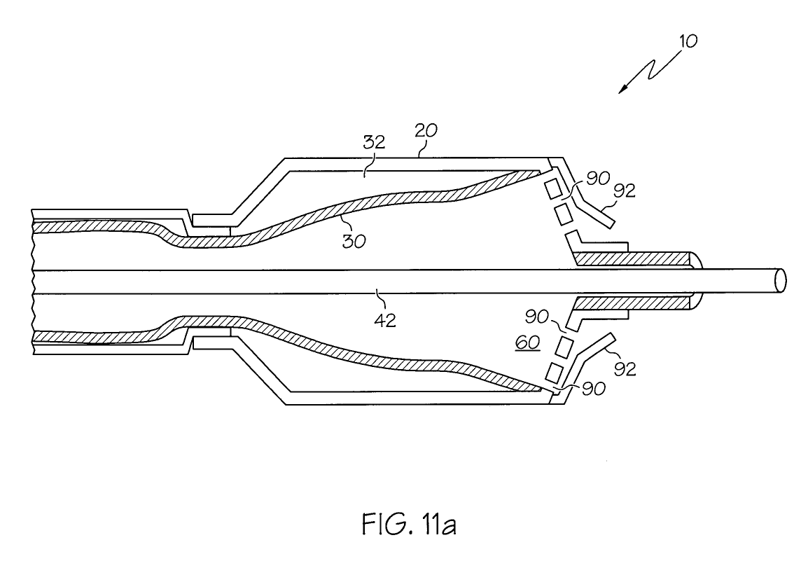

In some embodiments of the invention, the coolant ports 90 may be provided with valves, baffles, barriers and/or other mechanisms which permit outflow of the infusate while preventing backflow of the fluid or other bodily fluids. In FIG. 11 the coolant ports 90 are provided with baffles 92. The baffles 92 yield more readily to flow from within the guidewire lumen 60 than to backflow from outside the catheter. Though not represented in FIG. 11, it should be noted that baffles may be applied to each individual port 90 rather than one baffle covering several ports. In FIG. 11a the inner shaft 30 is in an expanded state. When in the expanded state infusate may be delivered through the ports 90 with less infusate pressure and thus less jetting of the infusate into the body lumen than when in the unexpanded state.

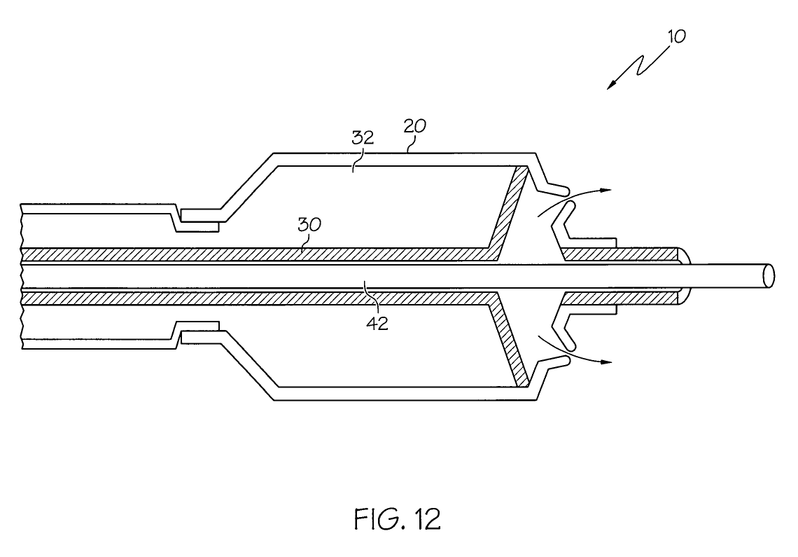

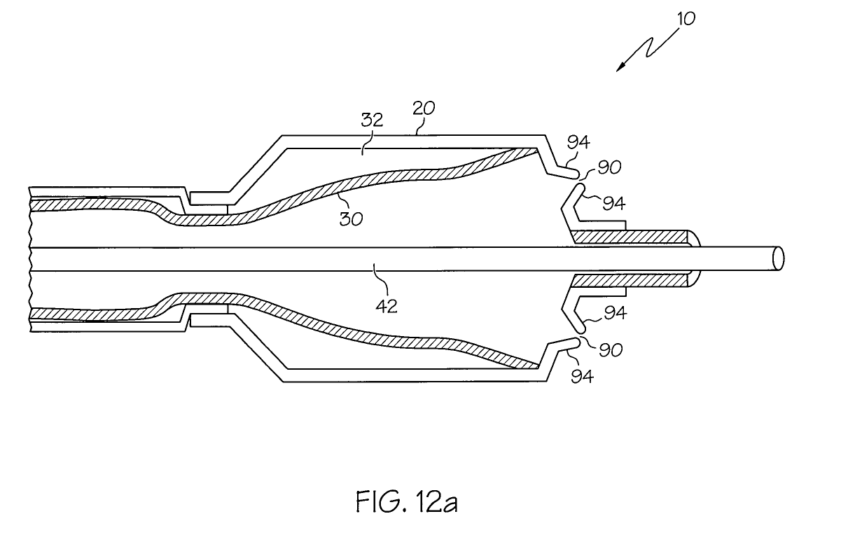

In some embodiments as shown in FIG. 12 the coolant ports 90 are provided with valves 94. The valves 94 yield more readily to flow from within the guidewire lumen 60 than to backflow from outside the catheter. In FIG. 12a the inner shaft 30 is in an expanded state. When in the expanded state infusate may be delivered through the ports 90 with less infusate pressure and thus less jetting of the infusate into the body lumen than when in the unexpanded state.

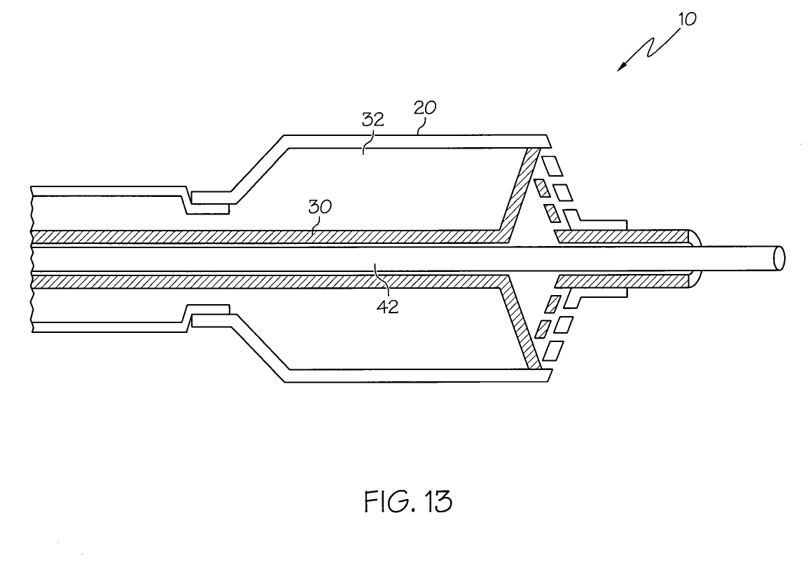

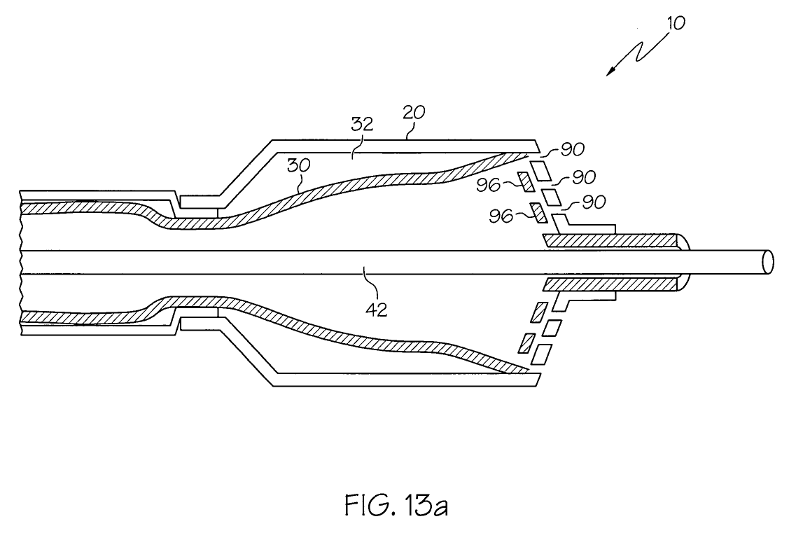

In some embodiments as shown in FIG. 13 the coolant ports 90 are provided with barriers 96. The barriers 96 yield more readily to flow from within the guidewire lumen 60 than to backflow from outside the catheter. In some embodiments the barriers allow fluids with certain viscosities and/or cohesion forces easier exit through the ports 90 than the backflow of different fluids from outside the catheter. In FIG. 13a the inner shaft 30 is in an expanded state. When in the expanded state infusate may be delivered through the ports 90 with less infusate pressure and thus less jetting of the infusate into the body lumen than when in the unexpanded state.

In at least one embodiment the infusate has a predetermined viscosity that is less than the viscosity of the blood and/or other fluids typically present in the vessel 36. The openings within the cone and inner shaft are then sized to allow passage of a fluid having a viscosity substantially equal or less than that of the infusate but not fluids having a greater viscosity than the infusate. In at least one embodiment the openings are sized and/or configured to allow fluids having a viscosity similar to that of water and/or saline to pass therethrough, or approximately 1-2 centipoises. In some embodiments the coolant ports 90 are about 8 microns to about 75 microns in area.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

1. A catheter comprising:
an outer shaft and an inner shaft, the outer shaft and inner shaft defining a balloon inflation lumen for transport of an inflation fluid therethrough,
an expandable balloon having a proximal waist portion and a distal waist portion with a body portion there between, the balloon being engaged to a distal region of the outer shaft, the balloon defining an exterior and an interior, the interior being in fluid communication with the balloon inflation lumen
the inner shaft having an inner shaft wall defining a guidewire lumen for passage of a guidewire, a distal portion of the inner shaft being expandable from an unexpanded state to an expanded state, in the expanded state the guidewire lumen having a greater cross-sectional area than in the unexpanded state.

2. The catheter of claim 1 including a guidewire along the entire length of the catheter.

3. The catheter of claim 2 wherein when in the expanded state the inner shaft is constructed and arranged to release an infusate onto tissue around the distal end of the catheter.

4. The catheter of claim 3 wherein the distal portion of the guidewire lumen has an expanded position and an unexpanded position, a predetermined infusate flow rate through the guidewire lumen is attained with less pressure when in the expanded position than when in the unexpanded position.

5. The catheter of claim 4 wherein when the distal portion of guidewire lumen is in the unexpanded position, the guidewire lumen has a diameter of between 0.010 and 0.017 inches.

6. The catheter of claim 4 wherein when the distal portion of the guidewire lumen is in the expanded position, the guidewire lumen has a diameter of between 0.018 and 0.025 inches.

7. The catheter of claim 1 wherein a distal portion of the inner shaft is constructed of deformable material that is unfoldable with infusate pressure.

8. The catheter of claim 7 wherein the foldable material is ePTFE.

9. The catheter of claim 2 wherein a distal portion of the inner shaft is constructed of a flexible or elastic material that radially stretches with infusate pressure.

10. The catheter of claim 9 wherein the elastic material is selected from the group of elastomeric materials consisting of polyurethane, SIBS, or any combination thereof.

11. The catheter of claim 1 wherein the distal portion of the inner shaft which is expandable is constructed of a material that shortens as it expands radially.

12. The catheter of claim 11 wherein the distal portion of the inner shaft which is expandable is constructed of a mesh having an expanding cover which separates the mesh from the balloon inflation lumen.

13. The catheter of claim 11 wherein longitudinal pressure applied distally to a proximal portion of the inner shaft will radially expand the distal portion of the inner shaft which is expandable.

14. The catheter of claim 13 wherein the inner shaft is affixed to the balloon at a point distal to the body portion of the balloon such that beyond this point the inner shaft does not substantially move in relation to the outer shaft.

15. The catheter of claim 1 wherein the expandable distal portion of the inner shaft is constructed and arranged to expand proximal to the proximal waist portion of the balloon and within the outer shaft.

16. The catheter of claim 1 wherein the expandable distal portion of the inner shaft is constructed and arranged to expand distal to the proximal waist portion of the balloon.

17. The catheter of claim 1 wherein the inner shaft is attached to the distal cone portion of the balloon, the distal cone portion having coolant ports constructed and arranged to allow release of an infusate.

18. The catheter of claim 1 wherein the infusate is a fluid having a temperature of less than about 37 Celsius.

19. The catheter of claim 1 wherein at least a portion of the distal waist portion is an extension of the inner shaft.

20. The catheter of claim 19 wherein the at least a portion of the distal waist portion has coolant ports which allow release of an infusate.

21. The catheter of claim 20 wherein the coolant ports are constructed and arranged such that infusate will not substantially pass through the coolant ports before the expandable portion of the inner shaft is expanded.

22. The catheter of claim 1 wherein the infusate is a fluid having a temperature of about 33 degrees Celsius to about 37 degrees Celsius.

23. The catheter of claim 19 wherein at least a portion of the distal waist portion comprises a plurality of coolant ports or openings, the ports constructed and arranged to allow the guidewire lumen during expansion to build pressure while allowing a sufficient outflow of infusate to adequately cool the surrounding tissue.

24. The catheter of claim 3 wherein the infusate has a viscosity less than that of blood.

25. The catheter of claim 3 wherein the infusate is selected from the group of fluids consisting of saline, Ringer Lactate solution, or any combination thereof.

26. The catheter of claim 20 wherein the coolant ports are configured to allow the infusate to exit the guidewire lumen under pressure but prevent or restrict the flow of bodily fluids into the guidewire lumen during deflation.

27. The catheter of claim 1 wherein the catheter is configured for the delivery of one or more therapeutic agents.

28. The catheter of claim 3 wherein a therapeutic agent is included with the infusate.

29. A method of cooling tissue within a body vessel comprising the following steps:
Inserting the device of claim 1 into a body lumen at a treatment site,
partially inflating the balloon,
introducing infusate into the inner shaft, the infusate under pressure such that the inner shaft expands and infusate is delivered to the tissue being treated.

30. A catheter comprising:
an outer shaft and an inner shaft, the inner shaft having an inner shaft wall defining a guidewire lumen for passage of a guidewire, a distal portion of the inner shaft being expandable from an unexpanded state to an expanded state, in the expanded state the guidewire lumen having a greater cross-sectional area than in the unexpanded state.
